(19) United States
(12) Patent Application Publication
(10) Pub. No.: US 2020/0392145 A1
(43) Pub. Date: Dec. 17, 2020

(54) PROCESS

(71) Applicant: Johnson Matthey Public Limited Company, London (GB)

(72) Inventors: Nicolas ARCHER, Edinburgh (GB); Michael BEASE, Edinburgh (GB); Rebecca FISHER, Glasgow (GB); Susan ANDREWS, Edinburgh (GB)

(73) Assignee: Johnson Matthey Public Limited Company, London (GB)

(21) Appl. No.: 16/767,400

(22) PCT Filed: Jan. 18, 2018
(86) PCT No.: PCT/GB2018/050137

§ 371 Date: May 27, 2020

(30) Foreign Application Priority Data
Nov. 27, 2017 (GB) 1719667.6

(51) Int. Cl. C07D 489/12

(52) U.S. Cl. CPC C07D 489/12

(57) ABSTRACT

The present invention provides a process for the preparation of a compound of formula (2): the process comprising reacting a compound of formula (1), a base and an alkylating agent R₄—X in a nitrile-containing polar aprotic solvent to form the compound of formula (2), wherein the process is carried out at a temperature greater than 60° C.; and wherein R₁, R₂, R₃, R₄, and X are as defined in the specification.

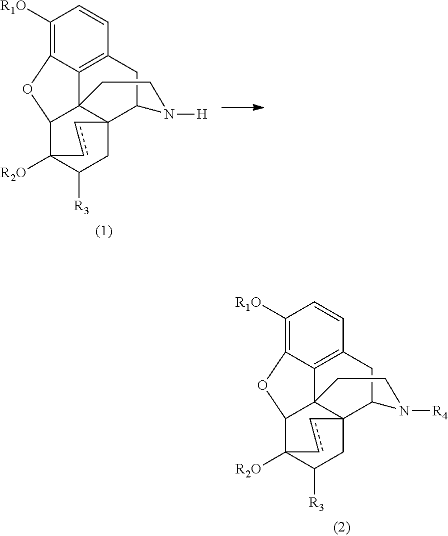

The present invention provides a process for the production of morphinan alkaloids. In particular, the invention provides an improved process for the production of morphinan alkaloids substituted at N-17 with a group other than methyl.

### Definitions

The point of attachment of a moiety or substituent is represented by “-”. For example, —OH is attached through the oxygen atom.

“Alkyl” refers to a straight-chain or branched saturated hydrocarbon group. In certain embodiments, the alkyl group may have from 1-20 carbon atoms, in certain embodiments from 1-15 carbon atoms, in certain embodiments, 1-8 carbon atoms. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert-butyl, n-pentyl, n-hexyl, and the like.

The term “cycloalkyl” is used to denote a saturated carbocyclic hydrocarbon radical. The cycloalkyl group may have a single ring or multiple condensed rings. In certain embodiments, the cycloalkyl group may have from 3-15 carbon atoms, in certain embodiments, from 3-10 carbon atoms, in certain embodiments, from 3-8 carbon atoms. The cycloalkyl group may be unsubstituted. Alternatively, the cycloalkyl group may be substituted. Unless other specified, the cycloalkyl group may be attached at any suitable carbon atom and, if substituted, may be substituted at any suitable atom. Typical cycloalkyl groups include but are not limited to cyclopropyl, cyclobutyl, cyclopentyl, cyclohexyl and the like.

“Aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted or substituted. Unless otherwise specified, the aryl group may be attached at any suitable carbon atom and, if substituted, may be substituted at any suitable atom. Examples of aryl groups include, but are not limited to, phenyl, naphthyl, anthracenyl and the like.

“Arylalkyl” refers to an optionally substituted group of the formula aryl-alkyl-, where aryl and alkyl are as defined above.

“Halo” or “halogen” refers to —F, —C, —Br and —I e.g. —Cl, —Br and —I.

“Morphinan” refers to a compound comprising the core structure:

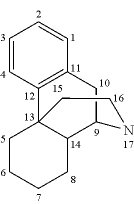 [SMILES: c1ccc2c(c1)CC1NCCC23CCCCC13]

“Substituted” refers to a group in which one or more (e.g. 1, 2, 3, 4 or 5) hydrogen atoms are each independently replaced with substituents which may be the same or different. The substituent may be any group which tolerates the alkylation reaction conditions. Examples of substituents include but are not limited to —R^{a}, —O—R^{a}, —S—R^{a}, —NR^{a}R^{b} and —NHR^{a}; wherein R^{a} and R^{b} are independently selected from the groups consisting of alkyl, cycloalkyl, aryl and arylalkyl. R^{a} and R^{b} may be unsubstituted or further substituted as defined herein.

### DETAILED DESCRIPTION

The present invention provides a process for the preparation of a compound of formula (2):

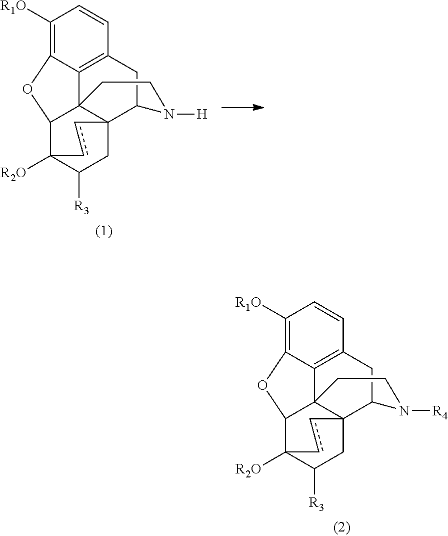

the process comprising reacting a compound of formula (1), a base and an alkylating agent R₄—X in a nitrile-containing polar aprotic solvent to form the compound of formula (2), wherein the process is carried out at a temperature greater than 60° C.; and
wherein:
R₁ and R₂ are independently selected from the group consisting of —H, an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl and alcohol protecting group;
R₃ is —C(R₁₀)(R₁₁)(OH) or a protected —C(═O)(R₁₂);
R₄ is selected from the group consisting of an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl, unsubstituted —C₁₋₂₀-alkyl-C₃₋₂₀-cycloalkyl, substituted —C₁₋₂₀-alkyl-C₃₋₂₀-cycloalkyl, unsubstituted allyl and substituted allyl;
R₁₀, R₁₁ and R₁₂ are independently selected from the group consisting of an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl and substituted cyclic C₃-C₂₀-alkyl; [symbol]
is a double bond or a single bond; and
X is a halo group.

The compounds described herein may have chiral centres at positions C-5, C-6, C-7, C-9, C-13 and C-14 of the morphinan structure. The ethano/ethano bridge between carbon atoms C-6 and C-14 is either on the alpha or beta face of the compound. The compounds of formulae (1) and (2) may have the stereochemistry shown below:

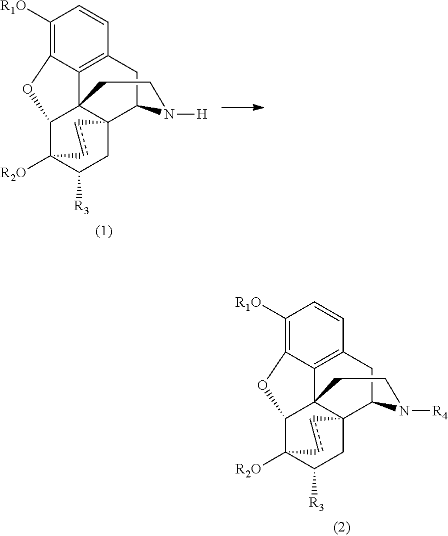

When R₁ and/or R₂ are H, the hydroxy groups present at C-3- and/or C-6 may be susceptible to alkylation. Thus, it is may be desirable to first protect one or both of the hydroxy groups with a suitable protecting group which may be optionally removed after the alkylation is completed. Protecting groups are known in the art and methods for their introduction and removal are described in standard references such as “Greene's Protective Groups in Organic Synthesis”, P. G. M. Wuts and T. W. Greene, 4th Edition, Wiley.

R₁ is selected from the group consisting of —H, an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl and alcohol protecting group. R₁ may be selected from the group consisting of —H, an unsubstituted straight-chain C₁-C₂₀-alkyl, unsubstituted branched-chain C₁-C₂₀-alkyl, and unsubstituted cyclic C₃-C₂₀-alkyl. R₁ may be selected from the group consisting of —H and an unsubstituted straight-chain C₁-C₂₀-alkyl, such as —H or -Me. In one embodiment, R₁ may be —H. In another embodiment, R₁ may be -Me.

R₂ is selected from the group consisting of —H, an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl and alcohol protecting group. R₂ may be selected from the group consisting of —H, an unsubstituted straight-chain C₁-C₂₀-alkyl, unsubstituted branched-chain C₁-C₂₀-alkyl, and unsubstituted cyclic C₃-C₂₀-alkyl. R₂ may be selected from the group consisting of —H and an unsubstituted straight-chain C₁-C₂₀-alkyl, such as —H or -Me. In one embodiment, R₂ may be —H. In another embodiment, R₂ may be -Me.

One of R₁ and R₂ may be selected from the group —H and the other of R₁ and R₂ may be an unsubstituted straight-chain C₁-C₂₀-alkyl. For example, one of R₁ and R₂ may be —H and the other of R₁ and R₂ may be -Me. R₁ may be —H or -Me and R₂ may be -Me.

R₃ may be —C(R₁₀)(R₁₁)(OH), wherein R₁₀ and R₁₁ are independently selected from the group consisting of an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl and substituted cyclic C₃-C₂₀-alkyl.

R₁₀ may be selected from the group consisting of an unsubstituted straight-chain C₁-C₂₀-alkyl, unsubstituted branched-chain C₁-C₂₀-alkyl, and unsubstituted cyclic C₃-C₂₀-alkyl. For example, R₁₀ may be selected from a butyl (i-, p- or b-) and a methyl group. R₁₀ may be a tert-butyl or methyl group.

R₁₁ may be selected from the group consisting of an unsubstituted straight-chain C₁-C₂₀-alkyl, unsubstituted branched-chain C₁-C₂₀-alkyl, and unsubstituted cyclic C₃-C₂₀-alkyl. For example, R₁₁ may be selected from a propyl (n- or i-), butyl (n-, i-, p- or t-) or a methyl group. R₁₁ may be a n-propyl, tert-butyl or methyl group.

In one embodiment, R₃ is

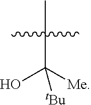 [SMILES: *C(C)(O)C(C)(C)C]

In another embodiment, R₃ is

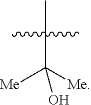 [SMILES: *C(C)(C)O]

In another embodiment, R₃ is

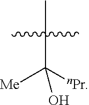

R₃ may be a protected —C(═O)(R₁₂). It is may be desirable to first protect the keto group with a suitable protecting group which may be optionally removed after the alkylation step is completed. Protecting groups are known in the art and methods for their introduction and removal are described in standard references such as “Greene's Protective Groups in Organic Synthesis”, P. G. M. Wuts and T. W. Greene, 4^{th} Edition, Wiley. Suitable keto protecting groups include but are not limited to acetals and ketals. For example, substituted or unsubstituted, straight-chain or branched C₁-C₂₀-alkanols, substituted or unsubstituted, straight-chain or branched 1,2-(C₁-C₂₀)-alkyl-diols (for example, ethylene glycol or 1,2-propanediol), or substituted or unsubstituted, straight-chain or branched 1,3-(C₁-C₂₀)-alkyldiols may be conveniently utilised to form suitable acetals or ketals. A diol reacts to form a ring and in this instance, the ketal comprises substituted or unsubstituted chiral or achiral bridges which are derived, for example, from the skeletons —(CH₂)ₙ— (n=2, 3 or 4), —CH(CH₃)CH(CH₃)—, —CH(CH₃)CH₂CH(CH₃)—, —CMe₂-, —CHMe-, no limitation being implied by this listing. The protecting group may be removed by methods known in the art to form C(═O)(R₁₂).

R₁₂ may be selected from the group consisting of an unsubstituted straight-chain C₁-C₂₀-alkyl, unsubstituted branched-chain C₁-C₂₀-alkyl, and unsubstituted cyclic C₃-C₂₀-alkyl. For example, R₁₂ may be a methyl group.

[symbol] is a double bond or a single bond. In one embodiment, [symbol] is a —C═C— double bond. In another embodiment, [symbol] is a —C—C— single bond.

The compound of formula (1) may be:

| | R₁ | R₂ | R₃ | [symbol] | Name |
|---|---|---|---|---|---|
| i) | —H | —Me | 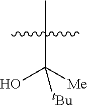 [SMILES: *C(C)(O)C(C)(C)C] | —C—C— single bond | 6,14-endo-ethano-7-(2- hydroxy-3,3-dimethyl)- tetrahydronororipavine [i.e. Norbuprenorphine]; |
| ii) | —H | —Me | 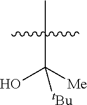 [SMILES: *C(C)(O)C(C)(C)C] | —C═C— double bond | 6,14-endo-etheno-7-(2- hydroxy-3,3-dimethyl)- tetrahydronororipavine; |
| iii) | —Me | —Me | 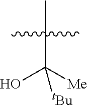 [SMILES: *C(C)(O)C(C)(C)C] | —C—C— single bond | 6,14-endo-ethano-7-(2- hydroxy-3,3-dimethyl)- tetrahydronorthebaine [i.e. 3-O-Methyl- norbuprenorphine]; |
| iv) | —Me | —Me | 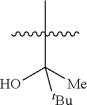 [SMILES: *C(C)(O)C(C)(C)C] | —C═C— double bond | 6,14-endo-etheno-7-(2- hydroxy-3,3-dimethyl)- tetrahydronorthebaine; |
| v) | —H | —Me | 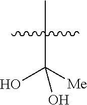 [SMILES: *C(C)(O)O] | —C—C— single bond | 6,14-endo-ethano-7-(2- hydroxy-2-propyl)- tetrahydronororipavine [i.e. Nordiprenorphine]; |
| vi) | —H | —Me | 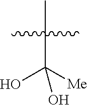 [SMILES: *C(C)(O)O] | —C═C— double bond | 6,14-endo-etheno-7-(2- hydroxy-2-propyl)- tetrahydronororipavine; |
| vii) | —Me | —Me | 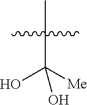 [SMILES: *C(C)(O)O] | —C—C— single bond | 6,14-endo-ethano-7-(2- hydroxy-2-propyl)- tetrahydronorthebaine [i.e. 3-O-Methyl- nordiprenorphine]; |
| viii) | —Me | —Me | 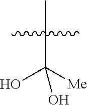 [SMILES: *C(C)(O)O] | —C═C— double bond | 6,14-endo-etheno-7-(2- hydroxy-2-propyl)- tetrahydronorthebaine. |

The compound of formula (2) may be:

| | R₁ | R₂ | R₃ | R₄ | [symbol] | Name |
|---|---|---|---|---|---|---|
| i) | —H | —Me | 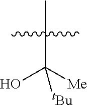 [SMILES: *C(C)(O)C(C)(C)C] | 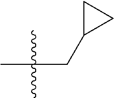 [SMILES: *CC1CC1] | —C—C— single bond | N-cydopropylmethyl- 6,14-endo-ethano-7-(2- hydroxy-3,3-dimethyl)- tetrahydronororipavine [i.e. Buprenorphine]; |
| ii) | —H | —Me | 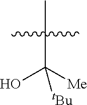 [SMILES: *C(C)(O)C(C)(C)C] | 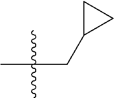 [SMILES: *CC1CC1] | —C═C— double bond | N-cyclopropylmethyl- 6,14-endo-etheno-7-(2- hydroxy-3,3-dimethyl)- tetrahydronororipavine; |
| iii) | —Me | —Me | 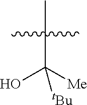 [SMILES: *C(C)(O)C(C)(C)C] | 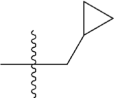 [SMILES: *CC1CC1] | —C—C— single bond | N-cydopropylmethyl- 6,14-endo-ethano-7-(2- hydroxy-3,3-dimethyl)- tetrahydronorthebaine [i.e. 3-O-Methyl- buprenorphine]; |
| iv) | —Me | —Me | 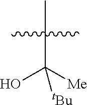 [SMILES: *C(C)(O)C(C)(C)C] | 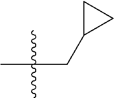 [SMILES: *CC1CC1] | —C═C— double bond | N-cyclopropylmethyl- 6,14-endo-etheno-7-(2- hydroxy-3,3-dimethyl)- tetrahydronorthebaine; |
| v) | —H | —Me | 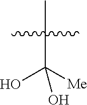 [SMILES: *C(C)(O)O] | 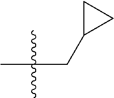 [SMILES: *CC1CC1] | —C—C— single bond | N-cyclopropylmethyl- 6,14-endo-ethano-7-(2- hydroxy-2-propyl)- tetrahydronororipavine; |
| vi) | —H | —Me | 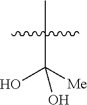 [SMILES: *C(C)(O)O] | 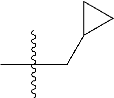 [SMILES: *CC1CC1] | —C═C— double bond | N-cyclopropylmethyl- 6,14-endo-etheno-7-(2- hydroxy-2-propyl)- tetrahydronororipavine; |
| vii) | —Me | —Me | 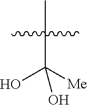 [SMILES: *C(C)(O)O] | 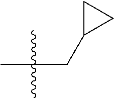 [SMILES: *CC1CC1] | —C—C— single bond | N-cyclopropylmethyl- 6,14-endo-ethano-7-(2- hydroxy-2-propyl)- tetrahydronorthebaine [i.e. 3-O-Methyl- diprenorphine]; or |
| viii) | —Me | —Me | 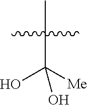 [SMILES: *C(C)(O)O] | 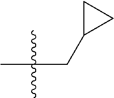 [SMILES: *CC1CC1] | —C═C— double bond | N-cyclopropylmethyl- 6,14-endo-etheno-7-(2- hydroxy-2-propyl)- tetrahydronorthebaine, |

The base may be an organic base or an inorganic base. When the base is an organic base, it may be selected from the group which includes but is not limited to amine bases, such as pyridine, triethylamine, tripropylamine, tributylamine, N,N-diisopropylamine, N-methylmorpholine, or N,N-dimethylaminopyridine.

When the base is an inorganic base, it may be selected from the group which includes but is not limited to borates, phosphates, acetates, carbonates and bicarbonates (i.e. hydrogen carbonates). Suitable borates include alkali metal borates (e.g. lithium borate, sodium borate or potassium borate). Suitable phosphates include alkali metal phosphates (e.g. lithium phosphate, sodium phosphate or potassium phosphate). Suitable acetates include alkali metal acetates (e.g. lithium acetate, sodium acetate or potassium acetate). Suitable carbonates include but are not limited to alkali metal carbonates (e.g. lithium carbonate, sodium carbonate or potassium carbonate) and alkaline earth metal carbonates (e.g. calcium carbonate). Suitable bicarbonates include but are not limited to alkali metal bicarbonates (e.g. lithium bicarbonate, sodium bicarbonate or potassium bicarbonate).

Strong bases, for example, hydroxides or alkoxides, may be used in the process of the present invention provided that hydroxy groups present at C-3 and/or C-6 of the compound of formula (1) (i.e. when R₁ and R₂ are —H) are protected beforehand with a suitable alcohol-protecting group. Examples of hydroxides include alkali metal hydroxides (e.g. lithium hydroxide, sodium hydroxide or potassium hydroxide) or tetraalkylammonium hydroxides. Examples of alkoxides include alkali metal alkoxides (e.g. lithium alkoxide, sodium alkoxide or potassium alkoxide) or tetraalkylammonium alkoxides.

The molar ratio of the compound (1):base may be from about 1:1 to about 1:2.0. In some embodiments, the molar ratio of the compound (1):base may be about 1:1. In some embodiments, the molar ratio of the compound (1):base may be about 1:1.1. In some embodiments, the molar ratio of the compound (1):base may be about 1:1.2. In some embodiments, the molar ratio of the compound (1):base may be about 1:1.3. In some embodiments, the molar ratio of the compound (1):base may be about 1:1.4. In some embodiments, the molar ratio of the compound (1):base may be about 1:1.5. In some embodiments, the molar ratio of the compound (1):base may be about 1:1.6. In some embodiments, the molar ratio of the compound (1):base may be about 1:1.7. In some embodiments, the molar ratio of the compound (1):base may be about 1:1.8. In some embodiments, the molar ratio of the compound (1):base may be about 1:1.9. In some embodiments, the molar ratio of the compound (1):base may be about 1:2.0.

The polar aprotic solvent has a nitrile (—C═N) group. The nitrile-containing aprotic solvent may have a boiling point at atmospheric pressure (i.e. 1.0135×10⁵ Pa) greater than 60° C. and below 250° C. The nitrile-containing aprotic solvent may be acetonitrile, propionitrile or butyronitrile. In one embodiment, nitrile-containing aprotic solvent is acetonitrile. It is desirable that the solvent is selected such that either compound (1) or compound (2) is partially soluble in the solvent i.e. the compound (1) or (2) is partially present as solid as well as being partially dissolved in the solvent. In this instance, the other of compound (1) or (2) is desirably substantially soluble in the solvent. For example, the compound (1) may be partially soluble in the solvent whereas the product, compound (2), may be substantially soluble in the solvent. Alternatively, the compound (1) may be substantially soluble in the solvent whereas the product, compound (2), may be partially soluble in the solvent. Without wishing to be bound by theory, it is believed that this difference in solubilities between starting material and product helps drive the alkylation reaction towards completion.

The ratio of compound (1):polar aprotic solvent may be in the range of about 0.01:0.5 g/mL. In some embodiments, the ratio of compound (1):solvent may be about 0.01 g/mL. In some embodiments, the ratio of compound (1):solvent may be about 0.02 g/mL. In some embodiments, the ratio of compound (1):solvent may be about 0.03 g/mL. In some embodiments, the ratio of compound (1):solvent may be about 0.04 g/mL. In some embodiments, the ratio of compound (1):solvent may be about 0.05 g/mL. In some embodiments, the ratio of compound (1):solvent may be about 0.06 g/mL. In some embodiments, the ratio of compound (1):solvent may be about 0.07 g/mL. In some embodiments, the ratio of compound (1):solvent may be about 0.5 g/mL. In some embodiments, the ratio of compound (1):solvent may be about 0.45 g/mL. In some embodiments, the ratio of compound (1):solvent may be about 0.40 g/mL. In some embodiments, the ratio of compound (1):solvent may be about 0.35 g/mL. In some embodiments, the ratio of compound (1):solvent may be about 0.20 g/mL. In some embodiments, the ratio of compound (1) solvent may be about 0.15 g/mL. In some embodiments, the ratio of compound (1) solvent may be about 0.10 g/mL. In some embodiments, the ratio of compound (1):solvent may be in the range of about 0.01 to 0.2 g/mL, such as about 0.06 to 0.10 g/mL, for example, about 0.08 g/mL.

The compound of formula (1), the base and the alkylating agent R₄—X are heated in the polar aprotic solvent to an internal temperature greater than 60° C. The temperature may be greater than 60° C. and up to the boiling point of the reaction mixture. The boiling point of the reaction mixture may vary depending on the pressure under which the alkylation reaction is conducted. The temperature may be in the range of >60° C. to about ≤250° C. In some embodiments, the temperature may be about ≥61° C. In some embodiments, the temperature may be about ≤62° C. In some embodiments, the temperature may be about ≥63° C. In some embodiments, the temperature may be about ≥64° C. In some embodiments, the temperature may be about ≤250° C. In some embodiments, the temperature may be about ≤240° C. In some embodiments, the temperature may be about ≤230° C. In some embodiments, the temperature may be about ≤220° C. In some embodiments, the temperature may be about ≤210° C. In some embodiments, the temperature may be about ≤200° C. In some embodiments, the temperature may be about ≤190° C. In some embodiments, the temperature may be about ≤180° C. In some embodiments, the temperature may be about ≤170° C. In some embodiments, the temperature may be about ≤160° C. In some embodiments, the temperature may be about ≤150° C. In some embodiments, the temperature may be about ≤140° C. In some embodiments, the temperature may be about ≤130° C. In some embodiments, the temperature may be about ≤120° C. In some embodiments, the temperature may be about ≤110° C. In some embodiments, the temperature may be about ≤100° C. In some embodiments, the temperature may be about ≤90° C. In some embodiments, the temperature may be about ≤80° C. In some embodiments, the temperature may be about ≤70° C. In some embodiments, the temperature may be in the range of about ≥60° C. to ≤70° C., such as about ≥63° C. to ≤67° C., such as about 65° C.

Without wishing to be bound by theory, it is believed that the nitrogen lone pair of 17N-H acts as a nucleophile and reacts with the alkylating agent R₄—X to form a quaternary group. The quaternary group is then deprotonated with the base to form the compound (2).

R₄ is selected from the group consisting of an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl, unsubstituted —C₁₋₂₀-alkyl-C₃₋₂₀-cycloalkyl, substituted —C₁₋₂₀-alkyl-C₃₋₂₀-cycloalkyl, unsubstituted allyl and substituted allyl. R₄ may be selected from the group consisting of an unsubstituted straight-chain C₁-C₂₀-alkyl, unsubstituted branched-chain C₁-C₂₀-alkyl, unsubstituted cyclic C₃-C₂₀-alkyl, unsubstituted —C₁₋₂₀-alkyl-C₃₋₂₀-cycloalkyl, and unsubstituted allyl. For example, R₄ may be a cyclopropylmethyl

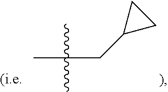 [SMILES: *CC1CC1]

cyclobutylmethyl

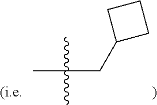 [SMILES: *CC1CCC1]

or allyl group

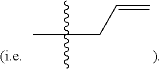 [SMILES: *CC=C]

In one embodiment, R₄ is a cyclopropylmethyl group.

X is a halo group which may be selected from —Cl, Br— or —I.

The molar ratio of the compound (1):R₄—X may be from about 1:1 to about 1:2.0. In some embodiments, the molar ratio of the compound (1) R₄—X may be about 1:1. In some embodiments, the molar ratio of the compound (1) R₄—X may be about 1:1.1. In some embodiments, the molar ratio of the compound (1) R₄—X may be about 1:1.2. In some embodiments, the molar ratio of the compound (1) R₄—X may be about 1:1.3. In some embodiments, the molar ratio of the compound (1) R₄—X may be about 1:1.4. In some embodiments, the molar ratio of the compound (1) R₄—X may be about 1:1.5. In some embodiments, the molar ratio of the compound (1) R₄—X may be about 1:1.6. In some embodiments, the molar ratio of the compound (1) R₄—X may be about 1:1.7. In some embodiments, the molar ratio of the compound (1) R₄—X may be about 1:1.8. In some embodiments, the molar ratio of the compound (1) R₄—X may be about 1:1.9. In some embodiments, the molar ratio of the compound (1):R₄—X may be about 1:2.0.

The alkylating agent R₄—X may be added to the compound (1) and the base in the polar aprotic solvent before the internal temperature of the reaction has reached >60° C. In this instance, the alkylating agent R₄—X may be added at the start of the process when the compound (1), base, and alkylating agent R₄—X are combined in the solvent. Alternatively, the compound (1), base and solvent may be heated to temperature (i.e. >60° C.) and the alkylating agent R₄—X added once the reaction mixture is at the desired temperature. The alkylating agent R₄—X may be added at a consistent rate (e.g. over a 30 minute time period or more) to control the alkylation at the 17N position. When R₁ is —H, a consistent addition rate also minimizes over alkylation at phenol group at C-3.

When X is —Br or —Cl, the process may further comprise an alkali metal iodide (e.g. sodium iodide or potassium iodide). Without wishing to be bound by theory, R₄—Cl or R₄—Br may undergo a halide exchange with the alkali metal iodide to form the corresponding R₄—I in situ. The initial reaction mixture therefore may comprise the compound (1), the base, the solvent, the alkali metal iodide, and either R₄—Cl or R₄—Br. The alkali metal iodide may be present in sub-stoichiometric, stoichiometric or greater than stoichiometric molar ratios as compared to the compound (1). The molar ratio of the compound (1):alkali metal iodide may be from about 1:1 to about 1:2.0. In some embodiments, the molar ratio of the compound (1) alkali metal iodide may be about 1:1. In some embodiments, the molar ratio of the compound (1):alkali metal iodide may be about 1:1.1. In some embodiments, the molar ratio of the compound (1):alkali metal iodide may be about 1:1.2. In some embodiments, the molar ratio of the compound (1):alkali metal iodide may be about 1:1.3. In some embodiments, the molar ratio of the compound (1):alkali metal iodide may be about 1:1.4. In some embodiments, the molar ratio of the compound (1):alkali metal iodide may be about 1:1.5. In some embodiments, the molar ratio of the compound (1):alkali metal iodide may be about 1:1.6. In some embodiments, the molar ratio of the compound (1):alkali metal iodide may be about 1:1.7. In some embodiments, the molar ratio of the compound (1):alkali metal iodide may be about 1:1.8. In some embodiments, the molar ratio of the compound (1):alkali metal iodide may be about 1:1.9. In some embodiments, the molar ratio of the compound (1):alkali metal iodide may be about 1:2.0.

Examples of R₄—X include but are not limited to cyclopropylmethyl chloride, cyclopropylmethyl bromide, cyclopropylmethyl iodide, cyclobutylmethyl chloride, cyclobutylmethyl bromide, cyclobutylmethyl iodide, allyl chloride, allyl bromide and allyl iodide.

The process may be carried out under an inert atmosphere, such as under nitrogen or argon gas.

The process is carried out for a period of time until it is determined that the process is complete. Completion of the process may be determined by in-process analysis or other suitable method. Typically, the process is complete within about 24 hours.

On completion, the reaction vessel and its contents may be cooled to ambient temperature and the solvent removed (for example, by distillation or stripping methods).

In another aspect, the present invention provides a process for the preparation of a compound of formula (4):

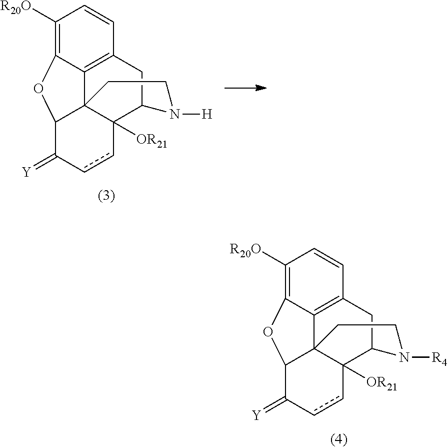

the process comprising reacting a compound of formula (3), a base and an alkylating agent R₄—X in a nitrile-containing polar aprotic solvent to form the compound of formula (4), wherein the process is carried out at a temperature greater than 60° C.; and
wherein:

### Y is a

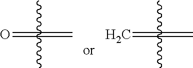

group;
R₂₀ and R₂₁ are independently selected from the group consisting of —H, an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl and alcohol protecting group;
R₄ is selected from the group consisting of an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl, unsubstituted C₁₋₂₀-alkyl-C₃₋₂₀-cycloalkyl, substituted C₁₋₂₀-alkyl-C₃₋₂₀-cycloalkyl, unsubstituted allyl and substituted allyl; [symbol]
is a double bond or a single bond; and
X is a halo group.

The alkylation conditions, base, alkylating agent R₄—X, nitrile-containing polar aprotic solvent, temperature, [symbol] , alkali metal iodide (if any), molar ratio of starting material:base, molar ratio of starting material:R₄—X, molar ratio of starting material:alkali metal iodide as described above for the first aspect of the invention generally likewise apply to this aspect of the invention.

The compounds described herein may have chiral centres at positions C-5, C-9, C-13 and C-14 of the morphinan structure. The compounds of formulae (3) and (4) may have the stereochemistry shown below:

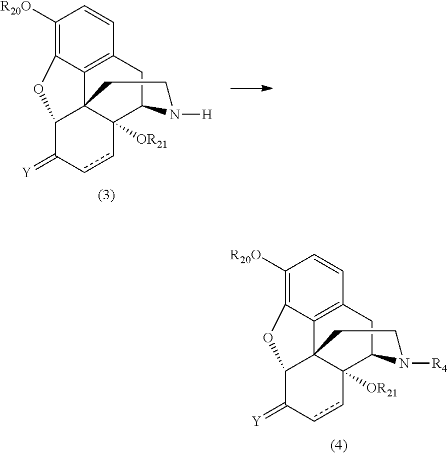

R₂₀ is selected from the group consisting of —H, an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl and alcohol protecting group. R₂₀ may be selected from the group consisting of —H, an unsubstituted straight-chain C₁-C₂₀-alkyl, unsubstituted branched-chain C₁-C₂₀-alkyl, and unsubstituted cyclic C₃-C₂₀-alkyl. R₂₀ may be selected from the group consisting of —H and an unsubstituted straight-chain C₁-C₂₀-alkyl, such as —H or -Me. In one embodiment, R₂₀ may be —H. In another embodiment, R₂₀ may be -Me.

R₂₁ is selected from the group consisting of —H, an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl and alcohol protecting group. R₂₁ may be selected from the group consisting of —H, an unsubstituted straight-chain C₁-C₂₀-alkyl, unsubstituted branched-chain C₁-C₂₀-alkyl, and unsubstituted cyclic C₃-C₂₀-alkyl. R₂₁ may be selected from the group consisting of —H and an unsubstituted straight-chain C₁-C₂₀-alkyl, such as —H or -Me. In one embodiment, R₂₁ may be —H. In another embodiment, R₂₁ may be -Me.

Y may be a

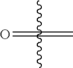 [SMILES: *=O]

group, which forms a carbonyl group with the carbon atom at C-6. Alternatively, Y can be a

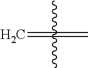

group, which forms an alkenyl group with the carbon atom at C-6.

The compound of formula (3) may be:

| | R₂₀ | R₂₁ | Y | [symbol] |
|---|---|---|---|---|
| i) | —H | —H | 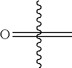 [SMILES: *=O] | —C—C— single bond; |
| ii) | —H | —H | 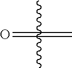 [SMILES: *=O] | —C═C— double bond; |
| iii) | —H | —H | 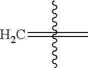 | —C—C— single bond; |
| iv) | —H | —H | 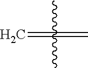 | —C═C— double bond; |
| v) | —Me | —H | 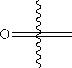 [SMILES: *=O] | —C—C— single bond; |
| vi) | —Me | —H | 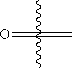 [SMILES: *=O] | —C═C— double bond; |
| vii) | —Me | —H | 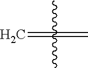 | —C—C— single bond; or |
| viii) | —Me | —H | 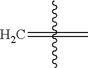 | —C═C— double bond. |

The compounds of formula (4) may be:

| | R₂₀ | R₂₁ | Y | R₄ | [symbol] |
|---|---|---|---|---|---|
| i) | —H | —H | 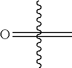 [SMILES: *=O] | 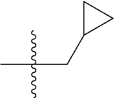 [SMILES: *CC1CC1] | —C—C— single bond; |
| ii) | —H | —H | 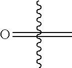 [SMILES: *=O] | 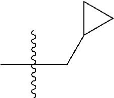 [SMILES: *CC1CC1] | —C═C— single bond; |
| iii) | —H | —H | 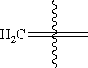 | 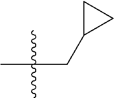 [SMILES: *CC1CC1] | —C—C— single bond; |
| iv) | —H | —H | 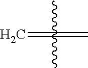 | 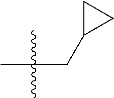 [SMILES: *CC1CC1] | —C═C— single bond; |
| v) | —Me | —H | 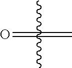 [SMILES: *=O] | 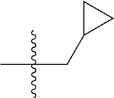 [SMILES: *CC1CC1] | —C—C— single bond; |
| vi) | —Me | —H | 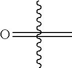 [SMILES: *=O] | 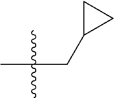 [SMILES: *CC1CC1] | —C═C— single bond; |
| vii) | —Me | —H | 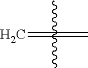 | 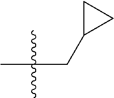 [SMILES: *CC1CC1] | —C—C— single bond; |
| viii) | —Me | —H | 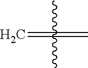 | 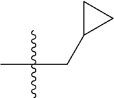 [SMILES: *CC1CC1] | —C═C— single bond; |
| ix) | —H | —H | 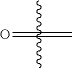 [SMILES: *=O] | 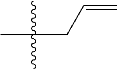 [SMILES: *CC=C] | —C—C— single bond; or |
| x) | —H | —H | 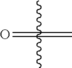 [SMILES: *=O] | 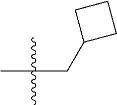 [SMILES: *CC1CCC1] | —C═C— single bond. |

Compounds (4) comprising

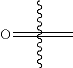 [SMILES: *=O]

as the Y group may be transformed into the

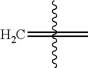

group by methods known in the art. For example, nalmefene may be prepared from naltrexone using methylenetriphenylphosphorane (Hahn et al, J. Med. Chem., 18, 259 (1975)).

Embodiments and/or optional features of the invention have been described above. Any aspect of the invention may be combined with any other aspect of the invention, unless the context demands otherwise. Any of the embodiments or optional features of any aspect may be combined, singly or in combination, with any aspect of the invention, unless the context demands otherwise.

The invention will now be described by way of the following non-limiting Example:

### EXAMPLE

### Example 1

The process is carried out under a nitrogen atmosphere.

Nordiprenorphine (1.3 g) is charged to a reaction vessel. Potassium bicarbonate (0.524 g), potassium iodide (0.87 g) and acetonitrile (15.6 mL) are added. The reaction mixture is heated to 65° C. while stirring. Cyclopropane methyl bromide (0.474 mL) is added slowly with a consistent addition rate over a 30 minute time period. Heating at 65° C. is continued for 13.5 hours. Stirring is stopped and the sediment is allowed to settle.

The suspension is allowed to cool to ambient temperature and transferred to a rotary evaporator flask. Acetonitrile may be used to aid the transfer. The suspension is concentrated to dryness using the rotary evaporator.

1. A process for the preparation of a compound of formula (2): 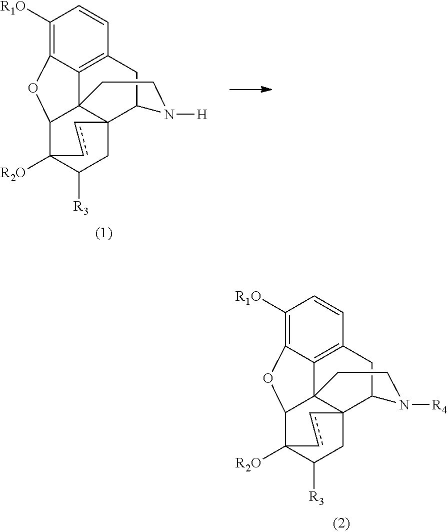
comprising reacting a compound of formula (1), a base and an alkylating agent R₄—X in a nitrile-containing polar aprotic solvent to form the compound of formula (2), wherein the process is carried out at a temperature greater than 60° C.; and
the compound of formula (1), the base and the nitrile-containing polar aprotic solvent are mixed to form a reaction mixture that is heated to the temperature greater than 60° C. and the alkylating agent R₄—X is added to the reaction mixture once the temperature is greater than 60° C.; and
wherein:
R₁ is —H;
R₂ is selected from the group consisting of —H, an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl and alcohol protecting group;
R₃ is —C(R₁₀)(R₁₁)(OH) or a protected —C(═O)(R₁₂);
R₄ is selected from the group consisting of an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl, unsubstituted —C₁₋₂₀-alkyl-C₃₋₂₀-cycloalkyl, substituted —C₁₋₂₀-alkyl-C₃₋₂₀-cycloalkyl, unsubstituted allyl and substituted allyl;
R₁₀, R₁₁ and R₁₂ are independently selected from the group consisting of an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl and substituted cyclic C₃-C₂₀-alkyl; [symbol]
is a double bond or a single bond; and
X is a halo group.

2. A process according to claim 1, wherein R₂ is selected from the group consisting of —H, an unsubstituted straight-chain C₁-C₂₀-alkyl, unsubstituted branched-chain C₁-C₂₀-alkyl, and unsubstituted cyclic C₃-C₂₀-alkyl.

3. A process according to claim 2, wherein R₂ is selected from the group consisting of —H and an unsubstituted straight-chain C₁-C₂₀-alkyl.

4. A process according to claim 1, wherein R₃ is —C(R₁₀)(R₁₁)(OH).

5. A process according to claim 1, wherein the compound of formula (1) is selected from the group consisting of.

| | R₁ | R₂ | R₃ | [symbol] |
|---|---|---|---|---|
| i) | —H | —Me | 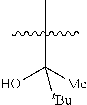 [SMILES: *C(C)(O)C(C)(C)C] | —C—C— single bond |
| ii) | —H | —Me | 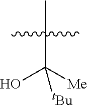 [SMILES: *C(C)(O)C(C)(C)C] | —C═C— double bond |
| v) | —H | —Me | 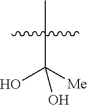 [SMILES: *C(C)(O)O] | —C—C— single bond |
| vi) | —H | —Me | 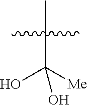 [SMILES: *C(C)(O)O] | —C═C— double bond |

6. A process according to claim 1, wherein the compound of formula (2) is selected from the group consisting of:

| | R₁ | R₂ | R₃ | R₄ | [symbol] |
|---|---|---|---|---|---|
| i) | —H | —Me | 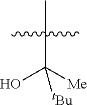 [SMILES: *C(C)(O)C(C)(C)C] | 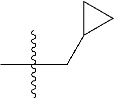 [SMILES: *CC1CC1] | —C—C— single bond; |
| ii) | —H | —Me | 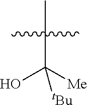 [SMILES: *C(C)(O)C(C)(C)C] | 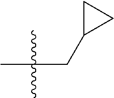 [SMILES: *CC1CC1] | —C═C— double bond; |
| v) | —H | —Me | 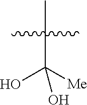 [SMILES: *C(C)(O)O] | 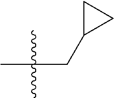 [SMILES: *CC1CC1] | —C—C— single bond; and |
| vi) | —H | —Me | 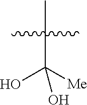 [SMILES: *C(C)(O)O] | 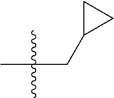 [SMILES: *CC1CC1] | —C═C— double bond. |

7. A process according to claim 1, wherein the base is an organic base or an inorganic base.

8. A process according to claim 7, wherein the inorganic base is selected from the group consisting of carbonates and bicarbonates.

9. A process according to claim 1, wherein the nitrile-containing aprotic solvent is selected from the group consisting of acetonitrile, propionitrile, and butyronitrile.

10. A process according to claim 1, wherein R₄ is selected from the group consisting of an unsubstituted straight-chain C₁-C₂₀-alkyl, unsubstituted branched-chain C₁-C₂₀-alkyl, unsubstituted cyclic C₃-C₂₀-alkyl, unsubstituted —C₁₋₂₀-alkyl-C₃₋₂₀-cycloalkyl, and unsubstituted allyl.

11. A process according to claim 10, wherein R₄ is selected from the group consisting of cyclopropylmethyl, cyclobutylmethyl and allyl.

12. A process according to claim 1, wherein the process further comprises an alkali metal iodide when R₄—X is R₄—Cl or R₄—Br.

13. A process for the preparation of a compound of formula (4): 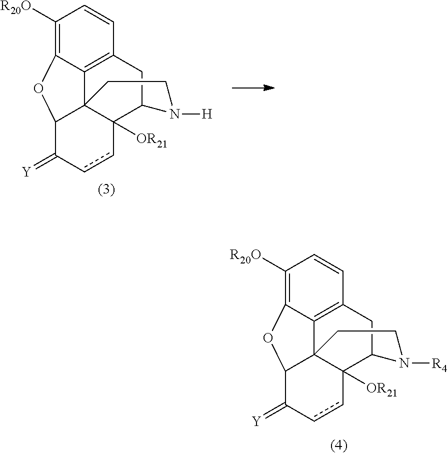
comprising reacting a compound of formula (3), a base and an alkylating agent R₄—X in a nitrile-containing polar aprotic solvent to form the compound of formula (4), wherein the process is carried out at a temperature greater than 60° C.; and
the compound of formula (3), the base and the nitrile-containing polar aprotic solvent are mixed to form a reaction mixture that is heated to the temperature greater than 60° C. and the alkylating agent R₄—X is added to the reaction mixture once the temperature is greater than 60° C.; and
wherein:
Y is a 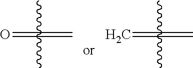
group;
R₂₀ is —H;
R₂₁ is selected from the group consisting of —H, an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl and alcohol protecting group;
R₄ is selected from the group consisting of an unsubstituted straight-chain C₁-C₂₀-alkyl, substituted straight-chain C₁-C₂₀-alkyl, unsubstituted branched-chain C₁-C₂₀-alkyl, substituted branched-chain C₁-C₂₀-alkyl, unsubstituted cyclic C₃-C₂₀-alkyl, substituted cyclic C₃-C₂₀-alkyl, unsubstituted —C₁₋₂₀-alkyl-C₃₋₂₀-cycloalkyl, substituted —C₁₋₂₀-alkyl-C₃₋₂₀-cycloalkyl, unsubstituted allyl and substituted allyl; [symbol]
is a double bond or a single bond; and
X is a halo group.

14. A process according to claim 13, wherein R₂₁ are is selected from the group consisting of —H, an unsubstituted straight-chain C₁-C₂₀-alkyl, unsubstituted branched-chain C₁-C₂₀-alkyl, and unsubstituted cyclic C₃-C₂₀-alkyl.

15. A process according to claim 14, wherein R₂₁ is selected from the group consisting of —H and an unsubstituted straight-chain C₁-C₂₀-alkyl.

16. A process according to claim 13, wherein the compound of formula (3) is selected from the group consisting of:

| | R₂₀ | R₂₁ | Y | [symbol] |
|---|---|---|---|---|
| i) | —H | —H | 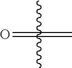 [SMILES: *=O] | —C—C— single bond; |
| ii) | —H | —H | 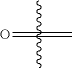 [SMILES: *=O] | —C═C— double bond; |
| iii) | —H | —H | 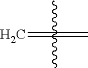 | —C—C— single bond; and |
| iv) | —H | —H | 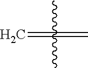 | —C═C— double bond. |

17. A process according to claim 13, wherein the compounds of formula (4) is selected from the group consisting of:

| | R₂₀ | R₂₁ | Y | R₄ | [symbol] |
|---|---|---|---|---|---|
| i) | —H | —H | 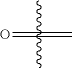 [SMILES: *=O] | 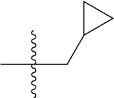 [SMILES: *CC1CC1] | —C—C— single bond; |
| ii) | —H | —H | 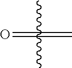 [SMILES: *=O] | 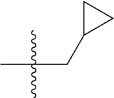 [SMILES: *CC1CC1] | —C═C— double bond; |
| iii) | —H | —H | 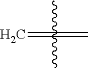 | 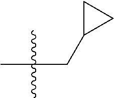 [SMILES: *CC1CC1] | —C—C— single bond; |
| iv) | —H | —H | 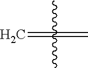 | 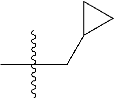 [SMILES: *CC1CC1] | —C═C— double bond; |
| iii) | —H | —H | 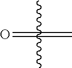 [SMILES: *=O] | 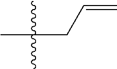 [SMILES: *CC=C] | —C—C— single bond; and |
| iv) | —H | —H | 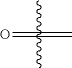 [SMILES: *=O] | 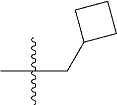 [SMILES: *CC1CCC1] | —C═C— double bond. |

18. The process according to claim 13, wherein the base is an organic base or an inorganic base.

19. A process according to claim 18, wherein the inorganic base is selected from the group consisting of carbonates and bicarbonates.

20. A process according to claim 13, wherein the nitrile-containing aprotic solvent is selected from the group consisting of acetonitrile, propionitrile, and butyronitrile.

21. A process according to claim 13, wherein R₄ is selected from the group consisting of an unsubstituted straight-chain C₁-C₂₀-alkyl, unsubstituted branched-chain C₁-C₂₀-alkyl, unsubstituted cyclic C₃-C₂₀-alkyl, unsubstituted C₁₋₂₀-alkyl-C₃₋₂₀-cycloalkyl, and unsubstituted allyl.

22. A process according to claim 21, wherein R₄ is selected from cyclopropylmethyl, cyclobutylmethyl or allyl.

23. A process according to claim 13, wherein the process further comprises an alkali metal iodide when R₄—X is R₄—Cl or R₄—Br.

24. A process according to claim 13, wherein the alkylating agent R₄—X is added at a consistent addition rate.

25. A process according to claim 1, wherein the alkylating agent R₄—X is added at a consistent addition rate.

26. A process according to claim 3, wherein R₂ is —H or -Me.

27. A process according to claim 15, wherein R₂₁ is —H or -Me.
